(19) United States
(12) Patent Application Publication
(10) Pub. No.: US 2001/0021705 A1
(43) Pub. Date: Sep. 13, 2001

(54) Disodium alendronate formulations

(75) Inventors: Gerald S. Brenner, Norristown, PA (US); Earl R. Oberholtzer JR., Hatfield, PA (US); J. Eric Thies, Scotch Plains, NJ (US)

Correspondence Address: Merck & Co., Inc., Patent Department, P.O. Box 2000 - RY60-30, Rahway, NJ, 07065-0907, US

(73) Assignee: MERCK & CO., Inc.

(21) Appl. No.: 09/841,126

(22) Filed: Apr. 24, 2001

Related U.S. Application Data
(63) Continuation of application No. 09/476,274, filed on Jan. 3, 2000.
Application No. 09/476,274 is a continuation of application No. 08/973,384, filed on Dec. 3, 1997.
Application No. 08/973,384 is a 371 of international application No. PCT/US96/08399, filed on Jun. 3, 1996.
Application No. PCT/US96/08399 is a continuation of application No. 08/469,142, filed on Jun. 6, 1995, now abandoned.

(51) Int. Cl. A61K 31/662; C07F 9/38

(52) U.S. Cl. 514/114; 562/12

(57) ABSTRACT

Disclosed is a method for treating and preventing bone loss in patients by administering a formulation of 4-amino-1-hydroxy-butylidene-1,1-bisphosphonic acid, disodium salt, or its hydrates. Also described is a pharmaceutical composition containing said disodium salt in a pharmaceutically acceptable excipient.

### FIELD OF THE INVENTION

The instant invention relates to the use of solid formulations of the disodium form of alendronate, i.e., 4-amino-1-hydroxy-butylidene-1,1-bisphosphonic acid disodium salt, and its hydrates, hereinafter referred to as “alendronate disodium” to inhibit bone resorption in human patients.

### BACKGROUND OF THE INVENTION

Normal bones are living tissues undergoing constant resorption and redeposition of calcium, with the net effect of maintenance of a constant mineral balance. The dual process is commonly called “bone turnover”. In normal growing bones, the mineral deposition is in equilibrium with the mineral resorption, whereas in certain pathological conditions, bone resorption exceeds bone deposition, for instance due to malignancy or primary hyperparathyroidism, or in osteoporosis. In other pathological conditions the calcium deposition may take place in undesirable amounts and areas leading to e.g., heterotopic calcification, osteoarthritis, kidney or bladder stones, atherosclerosis, and Paget's disease which is a combination of an abnormal high bone resorption followed by an abnormal calcium deposition.

U.S. Pat. No. 4,621,077 to Istituto Gentili discloses a method of treating urolithiasis and inhibiting bone reabsorption by the use of 4-amino-1-hydroxybutylidene-1,1-biphosphonic acid (also referred to as 4-amino-1-hydroxybutane-1,1-bisphosphonic acid) and its salts, with an alkali metal, an organic base or a basic amino acid. The compound 4-amino-1-hydroxybutylidene-1,1-biphosphonic acid is described as being between 100 and 300 times more active than dichloromethane-biphosphonic acid in inhibiting bone reabsorption.

Alendronate sodium, 4-amino-1-hydroxybutylidene-1,1-bisphosphonic acid monosodium trihydrate, is also an agent for combating bone resorption in bone diseases including osteoporosis and is described as a composition, method of use and synthesis along with other pharmaceutically acceptable salts in U.S. Pat. Nos. 4,922,007 and 5,019,651 (both assigned to Merck).

However, new crystalline salt forms of 4-amino-1-hydroxybutylidene-1,1-bisphosphonic acid are constantly being searched for to enable ease of formulation and better pharmacokinetics, e.g., desirable crystal habit, good flow properties, higher solubility, longer duration or quicker onset of action or improved bioavailability. Particularly what is desired is a new formulation to overcome the gastric irritability associated with the adminstration of the 4-amino-1-hydroxybutylidene-1,1-bisphosphonic acid in the free acid form. This is of particular importance in cases where the patient has a history of gastrointestinal problems prior to recommended alendronate therapy.

### SUMMARY OF THE INVENTION

The present invention provides a method for treating and/or preventing bone loss in a subject by the administering to said patient a pharmaceutically effective amount of the disodium form of alendronate, i.e., 4-amino-1-hydroxybutylidene-1,1-bisphosphonic acid disodium salt, or hydrates thereof. Because the aqueous pH of the disodium salt is about 8.7, as compared to the free acid which is about 2.2, there is substantially less gastric irritability associated with the disodium salt. This is particularly an important advance for patients with a history of gastrointestinal problems.

Also provided is a pharmaceutical composition comprising a pharmaceutically effective amount of alendronate disodium, i.e., 4-amino-1-hydroxybutylidene-1,1-bisphonic acid, disodium, or hydrates thereof, dispersed in a pharmaceutically acceptable excipient.

### DETAILED DESCRIPTION OF THE INVENTION AND PREFERRED EMBODIMENTS

The method disclosed herein can be used to treat humans, particularly females who are post-menopausal, with an osteogenically effective amount of alendronate disodium to inhibit bone resorption in need of such treatment. Such need arises locally in cases of bone fracture, non-union, defect, and the like. Such need also arises in cases of systemic bone disease, as in osteoporosis, osteoarthritis, Paget's disease, osteomalacia, multiple myeloma and other forms of cancer, steroid therapy, and age-related loss of bone mass.

The term “inhibition of bone resorption” as used herein, refers to treatment and prevention of bone loss, especially inhibiting the removal of existing bone either from the mineral phase and/or the organic matrix phase, through direct or indirect alteration of osteoclast formation or activity. Thus, the term “inhibitor of bone resorption” as used herein refers to agents that prevent bone loss by the direct or indirect alteration of osteoclast formation or activity and which may increase bone mass in patient treatment populations.

The term “osteogenically effective” as used herein, means that amount which effects the turnover of mature bone. As used herein, an osteogenically effective dose is also “pharmaceutically effective.”

The term “treatment” or “treating” as used herein shall mean (1) providing a subject with an amount of alendronate disodium sufficient to act prophylactically to prevent the development of a weakened and/or unhealthy state; and/or (2) providing a subject with a sufficient amount of alendronate disodium so as to alleviate or eliminate a disease state and/or the symptoms of a disease state, and a weakened and/or unhealthy state.

The term “hydrates” as used herein includes the hemihydrate, monohydrate, dihydrate, trihydrate, tetrahydrate, pentahydrate, hemipentahydrate (2.5 H₂O), and the like, of 4-amino-1-hydroxybutylidene-1,1-bisphosphonic acid disodium salt.

Pharmaceutical formulations of the invention which include alendronate disodium for administration will generally include an osteogenically effective amount of alendronate disodium to promote bone growth, in addition to a pharmaceutically acceptable excipient. The precise therapeutic dosage of alendronate disodium necessary will vary with the age, size, sex and condition of the subject, the nature and severity of the disorder to be treated, and the like; thus, a precise effective amount cannot be specified in advance and will be determined by the caregiver. However, appropriate amounts may be determined by routine experimentation with animal models, as described below. In general terms, an effective dose for alendronate disodium is about 0.01 to 1 mg/kg per day of body weight. Particularly useful dosages are 3.12, 6.24, 12.49 and 49.96 mg per day/per person of disodium alendronate monohydrate (equivalent to 2.5, 5.0, 10 and 40 mg free acid equivalents) per day per person.

The pharmaceutical composition described herein contains 4-amino-1-hydroxybutylidene-1,1-bisphosphonic acid disodium salt, in the anhydrous form or a hydrated form, in an amount of about 0.005 to 1.0 gram per gram of composition.

The pharmaceutical compositions according to the present invention containing alendronate disodium may be prepared for use in the form of capsules or tablets for oral administration or for systemic use. The compositions are advantageously prepared together with inert carriers such as sugars (saccharose, glucose, lactose), starch and derivatives, cellulose and derivatives, gums, fatty acids and their salts, polyalcohols, talc, aromatic esters, and the like.

The composition can also be prepared by direct compression of a dry mix formulation as described in U.S. Pat. No. 5,358,941 (assigned to Merck & Co. Inc.). Particularly useful diluents in the compostion are anhydrous lactose and microcrystalline cellulose.

Some typical pharmaceutical formulations (200 mg. oral tablets) containing 4-amino-1-hydroxybutylidene-1,1-diphosphonic acid disodium salt monohydrate, AHDPD, are shown below:

TABLETS (WHITE), 200 MG

| | COMPOSITION IN MG/TABLET | | | |
|---|---|---|---|---|
| | 2.5 | 5.0 | 10.0 | 40.0 |
| INGREDIENT | mg* | mg* | mg* | mg* |
| AHDPD** | 3.12 | 6.24 | 12.49 | 49.96 |
| Lactose Anydrous NF | 113.88 | 110.76 | 104.64 | 67.04 |
| Microcrystalline | 80.0 | 80.0 | 80.0 | 80.0 |
| Cellulose NF (Avicel | | | | |
| PH 102) | | | | |
| Magnesium Stearate NF | 1.00 | 1.00 | 1.00 | 1.00 |
| Croscarmellose Sodium | 2.00 | 2.00 | 2.00 | 2.00 |
| NF (Ac-Di-Sol) | | | | |
| Total | 200 | 200 | 200 | 200 |

*Anhydrous alendronate free acid equivalent, 4-amino-1-hydroxy-butylidene-1,1-bisphosphonic acid)
**AHDPD-4-amino-1-hydroxybutylidene-1,1-bisphosphonic acid disodium salt, monohydrate
Note: The amounts of inert ingredients can vary ±10%.

The methods and compositions of the invention are useful for treating bone fractures, defects and disorders which result from the pathological conditions of osteoporosis, osteoarthritis, Paget's disease, osteohalisteresis, osteomalacia, bone loss resulting from multiple myeloma other forms of cancer, bone loss resulting from side effects of disuse, other medical treatment (such as steroids), age-related and rheumatoid-related loss of bone mass.

The composition of the instant invention is also useful in lessening the risk of vertebral and non-vertebral fractures in osteoporotic post-menopausal women.

The composition described herein is also useful for the prevention and treatment of periodontal disease (see U.S. Pat. No. 5,270,365); to prevent or treat loosening of orthopedic implant devices; and, to lessen the risk in osteoporotic women of vertebral fractures, which composition can be administered in a protocol over a three year period.

The composition can also be used in combination with prostaglandins (see WO 94/06750), estrogen (see WO 94/14455), or growth hormone secretagogues to treat osteoporosis and the above-described conditions associated with abnormalities in bone resorption.

The following Examples are given to illustrate the carrying out of the invention as contemplated by the inventors and should not be construed as being limitations on the scope and spirit of the instantly described invention.

### EXAMPLE 1

4-Amino-1-Hydroxy-Butylidene-1,1-Bisphosphonic Acid Disodium Salt Monohydrate

To a suspension of 4-amino-1-hydroxy-1,1-diphosphonic acid (3.97 g) in 150 ml of distilled water was added with stirring aqueous sodium hydroxide (0.5N) until the pH of the soution was 9.2. The stirred solution was triturated with 200 ml ethanol (absolute) to give a suspension of a fine white solid which was chilled at 5 degrees C. overnight. The obtained solid was collected by vacuum filtration, air dried, and then dried in vacuo at 100 degrees C. at 0.2 torr for 18 hours over P₂O₅ to yield 4.38 g, (88%) yield of the disodium salt monohydrate title compound. A sample was submitted for CHN analysis;

For C₄H₁₁NO₄P₂Na₂:H₂O: Anal.: C, 15.44; H, 4.21; N, 4.50. Found: C, 15.28; H, 4.49; N, 4.49.

Melting Point of the solid was above 300 degrees C.

Solubility of the disodium salt in water is about 200mg/ml as compared to the free acid which is 8 mg/ml.

The solution pH of the disodium salt at 50 mg/ml. is 8.7, as compared to the free acid which is pH 2.2 at 8 mg/ml.

### EXAMPLE 2

Interconversion of Hydrated Forms

The above obtained monohydrate from Example 1 is exposed to a relative humidity atmosphere at 76% at room temperature for 24-48 hours resulting in the pentahydrate salt.

Exposure of this pentahydrate salt to 0% relative humidity at room temperature for 24-48 hours results in a trihydrate salt.

The trihydrate salt is heated to 100 degrees C. for 1-4 hours and results in a 2.5 hydrate (hemipentahydrate) salt.

The hemipentahydrate salt can be heated between 100-150 degrees C. for 1-4 hours to produce the hemihydrate.

The hemihydrate salt can be heated from 150-250 degrees C. for 1-4 hours to produce the anhydrous salt.

All of the above crystalline forms can be distinguished by their water content.

What is claimed is:

1. A pharmaceutical composition comprising a pharmaceutically effective amount of 4-amino-1-hydroxybutylidene-1,1-bisphosphonic acid disodium salt, or hydrates thereof, in a pharmaceutically acceptable carrier.

2. The pharmaceutical composition of claim 1 wherein said disodium salt, or hydrate thereof, is present in the amount of about 0.005 to 1.0 gram per gram of composition.

3. A method for treating and/or preventing bone loss in a subject, comprising administering to the subject in need thereof, the pharmaceutical composition as defined in claim 1.

4. The method of claim 3, wherein said subject is human.

5. The method of claim 3, wherein the bone loss is osteoporosis-related, due to disuse, age-related, related to steroid therapy, rheumatoid-related, related to Paget's disease, or related to cancer.

6. The method of claim 3, wherein the treatment is prophylactic.

7. 4-Amino-1-hydroxybutylidene-1,1-bisphosphonic acid disodium salt monohydrate.
